(19) United States
(12) Patent Application Publication
(10) Pub. No.: US 2013/0200254 A1
(43) Pub. Date: Aug. 8, 2013

(54) A PRESENCE DETECTION SYSTEM AND A LIGHTING SYSTEM

(71) Applicants: Mark Thomas Johnson, Arendonk (BE); Marieke Van Dooren, Arendonk (BE); Giovanni Cennini, Eindhoven (NL)

(72) Inventors: Mark Thomas Johnson, Arendonk (BE); Marieke Van Dooren, Arendonk (BE); Giovanni Cennini, Eindhoven (NL)

(73) Assignee: Koninklijke Philips Electronics N.V., Eindhoven (NL)

(21) Appl. No.: 13/879,865

(22) PCT Filed: Oct. 20, 2011
(86) PCT No.: PCT/IB11/54690

§ 371 Date: Apr. 17, 2013

(30) Foreign Application Priority Data
Oct. 27, 2010 (EP) 10189028.3

(51) Int. Cl. G01V 8/20; A61B 5/00

(52) U.S. Cl. CPC G01V 8/20; A61B 5/0059
USPC 250/208.2; 600/476; 600/473

(57) ABSTRACT

A presence detection system (1) for detecting a living being (10) within an area (12). The area (12) comprises at least two zones (12A;12B). The system (1) in total comprises at least two light sources (2A;2B) each of them having a different predefined spectrum and each of them radiating light in a different zone (12A;12B). The system comprises at least two sensing means (4A;4B) sensitive to light of the first predefined spectrum and the second predefined spectrum respectively. Each of the sensing means (4A;4B) is arranged for detecting light reflected from the living being (10) if present in the zone (12A;12B) of the area (12) and for generating a presence signal (14A;14B) based on detected light. The system comprises a processor device (8) that concludes the presence of the living being (10) within the area (12) in a zone wise way based on the presence signals. Such presence detection system provides relatively precise presence detection. The invention also relates to a lighting system comprising the above mentioned presence detection system (1).

### FIELD OF THE INVENTION

The invention relates to a presence detection system for detecting a living being, for example a human or an animal, within an area. The system comprises a light source for emitting light of a predefined spectrum, and a photo sensor, equipped with a spectral filter for filtering the light of the predefined spectrum. The photo sensor is arranged for detecting the light reflected from the living being present in the area which light passed the spectral filter and for generating a presence signal based on the detection result. The system further comprises a processor device for concluding the presence of the living being based on the presence signal.

The invention also relates to a lighting system comprising the above mentioned presence detection system.

### BACKGROUND OF THE INVENTION

An embodiment of such a system is disclosed in the patent application WO 97/26824. This patent application discloses an apparatus for monitoring a living body, for example a baby, involving transmitting radiation towards the body, receiving the radiation transmitted after modification by the body and processing the received radiation to determine the position and/or the state of the body, for example to determine if the body is breathing or its heart beating.

A drawback of the known system is the limited precision. Particularly, such a system is unable to precisely determine in which part of an area the living body is located.

### SUMMARY OF THE INVENTION

It is an object of the present invention to provide a presence detection system that is able to detect a living being, for example a human or an animal, within an area, the area comprising at least two zones, and to provide information in which of the zones the presence is detected. This object is achieved with the presence detection system for detecting a living being within an area according to the invention as defined in Claim 1. The system comprises a light source for emitting light of a predefined spectrum and a sensing means sensitive to light of the predefined spectrum. The sensing means is arranged for detecting the light reflected from the living being present in the area. The sensing means is arranged for generating a presence signal based on the detection result. The system further comprises a processor device for concluding the presence of the living being based on the presence signal. The area comprises in total at least two zones.

The system comprises in total at least two light sources each of them having a different predefined spectrum, a first spectrum and a second spectrum, and each of them radiating light in a different zone. The system comprises at least two sensing means sensitive to light of the first predefined spectrum and the second predefined spectrum respectively. Each of the sensing means is arranged for detecting light reflected from the living being if present in the zone of the area and for generating a presence signal based on detected light. The processor device concludes the presence of the living being within the area in a zone wise way based on the presence signals.

Differently from the system known in the art, the presence detection system according to the invention is able to determine in which zone of the area is the living being detected, i.e. the system provides a more precise presence detection result since the system concludes the presence of the living being in the zone wise way.

An embodiment of the system according to the invention has the feature that the sensing means comprises a photo sensor sensitive to the light of the predefined spectrum.

An embodiment of the system according to the invention has the feature that the sensing means comprises a photo sensor equipped with a spectral filter for filtering the light of predefined spectrum. The photo sensor is arranged for detecting light reflected from the living being if present in the area which detected light passed the spectral filter

An embodiment of the system according to the invention has the feature that the two light sources can be comprised in a single light-radiation device, which the light-radiation device is arranged to radiate lights of at least two different predefined spectrums in two zones. The first spectrum and the second spectrum can be substantially non-overlapping. However, even if these spectrums are overlapping, the presence detection system according to the invention will still work since each of the sensing means can be arrange to detect the light belonging to a first non-overlapping part of the first spectrum and a second non-overlapping part of the second spectrum.

An embodiment of the system according to the invention has the feature that the presence signal represents a vital signal of a human, for example a heart rate signal, a heart rate variation signal or a respiration rate signal, where the living being is the human. Such feature gives additional security that the human's presence is correctly concluded since the known heart rate signal's parameters, in case that the vital signal is the heart rate signal, can be used as additional criteria when concluding the human's presence.

An embodiment of the system according to the invention has the feature that the presence signal represents the vital signals of at least two humans present in a same zone within the area. The processor device is able to distinguish between the respective vital signals of the at least two humans present in the zone.

An embodiment of the system according to the invention has the feature that the predefined spectrum of light emitted by the light sources is a visible spectrum, for example within the range from 390 to 700 nanometers.

An embodiment of the system according to the invention has the feature that the predefined spectrum of light emitted by the light sources is an infrared spectrum, for example above 700 nanometers. Such feature provides an advantage that light emitted by the light sources of the presence detection system will not be visible to the humans. Such feature can be useful in an application where visible light can be disturbing. Since the system uses light of a non-visible spectrum range, the presence detection becomes completely unobtrusive.

An embodiment of the system according to the invention has the feature that each of the photo sensors comprises a photodiode and the photodiodes are together arranged in a photodiode array. Such a photodiode array is relatively cheap to manufacture. Each of the photodiodes is equipped with a spectral filter. Such spectral filters can be integrated with the respective photodiodes within the photodiode array.

The invention also relates to a lighting system, comprising the presence detection system as described in the previous embodiments and a further light source for illuminating one or more of the zones or illuminating the area. The presence detection system is arranged for controlling the further light sources or other systems such as security systems, acoustic, fragrance systems and haptic systems based on the presence signals.

### BRIEF DESCRIPTION OF THE DRAWINGS

In the following, the invention and further aspects will be described, by way of example, and explained hereinafter, using the following figures:

FIG. 1 schematically shows a first exemplary embodiment of a presence detection system wherein an area comprises two zones;

FIG. 2 schematically shows the intensity of presence signals as measured by the presence detection system according to the FIG. 1;

FIG. 3 schematically shows a second exemplary embodiment of a presence detection system wherein an area comprises three zones;

FIG. 4 schematically shows an absorption spectrum of the ohy-deoxyhemoglobin;

### DETAILED DESCRIPTION OF THE EMBODIMENTS

In the following description of the preferred embodiments, reference is made to the accompanying drawings which form a part thereof. Specific embodiments, in which the invention may be practiced, are shown in the following description by a way of illustration. It is also understood that other embodiments may be utilized and structural changes may be made without departing from the scope of the present invention. It is noted that the same reference signs will be used for indicating the same or similar parts in the several embodiments.

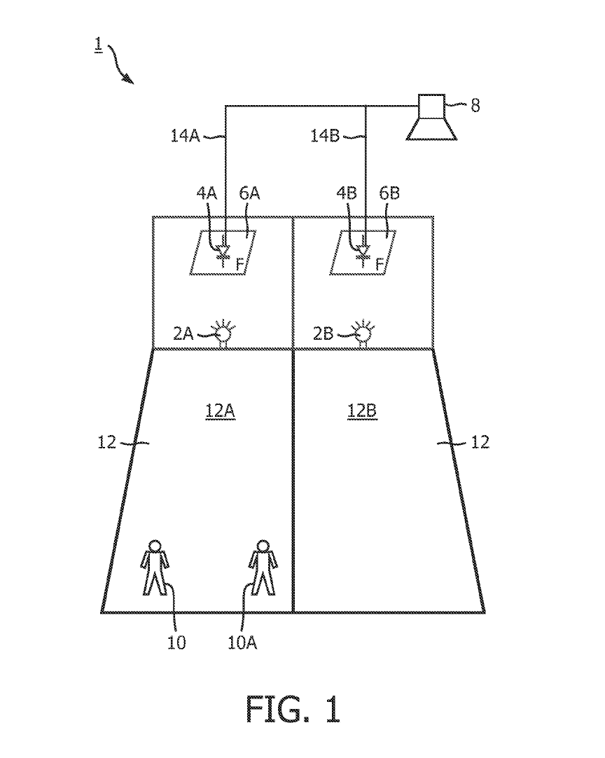

FIG. 1 schematically shows a first exemplary embodiment of the presence detection system according to the invention. In this embodiment, the system comprises two light sources, a first light source 2A and a second light source 2B, for emitting light of predefined spectrums and two sensing means, two photo sensors in this embodiment, a first photo sensor 4A and a second photo sensor 4B. In this embodiment, the photo sensors are equipped with spectral filters, a first spectral filter 6A and a second spectral filter 6B respectively, for filtering the light of the predefined spectra. The photo sensors 4A;4B are arranged for detecting light reflected from a first human 10 present in the area 12, which light passed the respective spectral filters 6A;6B, and for generating two presence signals, a first present signal 14A and a second presence signal 14B respectively, based on the detection results. The presence signals are provided to a processor device 8, which processor device concludes the presence or the absence of the first human 10 based on the provided presence signals 14A;14B. The area 12 is covered by two zones, a first zone 12A and a second zone 12B. Each of two light sources, the first light source 2A and the second light source 2B, has a different predefined spectrum and each of them radiates light within the different zone, the first zone 12A and the second zone 12B respectively. Each of two photo sensors, the first photo sensor 4A and the second photo sensor 4B, is arranged for working with one of the light sources, the first light source 2A and the second light source 2B respectively, and together with the corresponding spectral filter, the first spectral filter 6A and the second spectral filter 6B respectively, being arranged for the detection of the respective light of the predefined spectrum. In this way the processor device 8 concludes the presence of the first human 10 within the area 12 in a zone wise way.

Alternatively, the sensing means comprise photo sensors having different spectral sensitivities. Such photo sensor sensitive to the light of the predefined spectrum can be used instead of the photo sensor equipped with the spectral filter for filtering the light of predefined spectrum. The photo sensors must have different spectral sensitivities. In some embodiments, the photo sensors may have intrinsically different spectral sensitivities, whereby they can intrinsically distinguish between light of the different predefined spectra. The intrinsically different spectral sensitivities may be realized by e.g. using different classes of photo sensors, such as photodiodes, CCDs, photomultipliers etc. Alternatively the intrinsically different spectral sensitivities may be realized using photo sensors of the same class, such as photodiodes, with different material properties, e.g. Silicon (Si) based or Gallium arsenide (GaAs) based, different doping levels in Si etc. Alternatively the different spectral sensitivities can be realized by adding spectral filters.

In the concrete example shown in FIG. 1, the first human 10 is located in the first zone 12A within the area 12. Thus, only the first photo sensor 4A detects the light originated from the first light source 2A, reflected from the first human 10 and filtered by the first spectral filter 6A, while the second photo sensor 4B does not detect any significant light. This means that the processor device 8 receives two presence signals 14A;14B, wherein the first presence signal 14A indicates the presence of the human in the first zone 12A and the second presence signal 14B indicates the absence of the human in the second zone 12B. Consequently, the processor device 8 concludes the human's presence within the area 12 and more particularly the presence within the first zone 12A.

The photo sensors, for example photodiodes, are capable of measuring vital signals of the human. Photoplethysmography is the monitoring of variations of blood pulses in the dermis capillaries via absorption of light by oxy-deoxyhemoglobin. It is possible to perform photoplethysmography with simple photo sensors such as photodiodes. The photodiode monitors the tiny variations of the light intensity induced by the cyclic passage of fresh blood in the capillaries of the skin. With such a photodiode it is possible to dynamically measure the heart rate, heart rate variability, blood oxygenation and possibly blood pressure from a human at a distance of several meters. It is also known that the photodiodes are capable of measuring vital signals such as heart rate at a wide range of spectral frequencies. Most preferably, the system uses light of a non-visible spectrum range, whereby the measurement becomes completely unobtrusive. Thus, for example, the presence signals 14A;14B can represent the humans' heart rate signal. Such feature gives additional security that the humans' presence is correctly concluded since the known heart rate signal's parameters can be used as an additional criterion by the processor device when concluding the human's presence.

The spectral filters can be very simple components, for example thin film deposited, which are situated directly on top of the photo sensors, i.e. photodiodes. Alternatively, photodiodes or an array of photodiodes comprising the spectral filters, as known in the art, can be used. The photodiodes can be fabricated from standard Si semiconductor technology, or alternatively can be fabricated from amorphous Si technology which is used to fabricate LCD displays on glass or flexible, plastic substrates. The latter mentioned technology has the advantage of lower cost and larger area diodes.

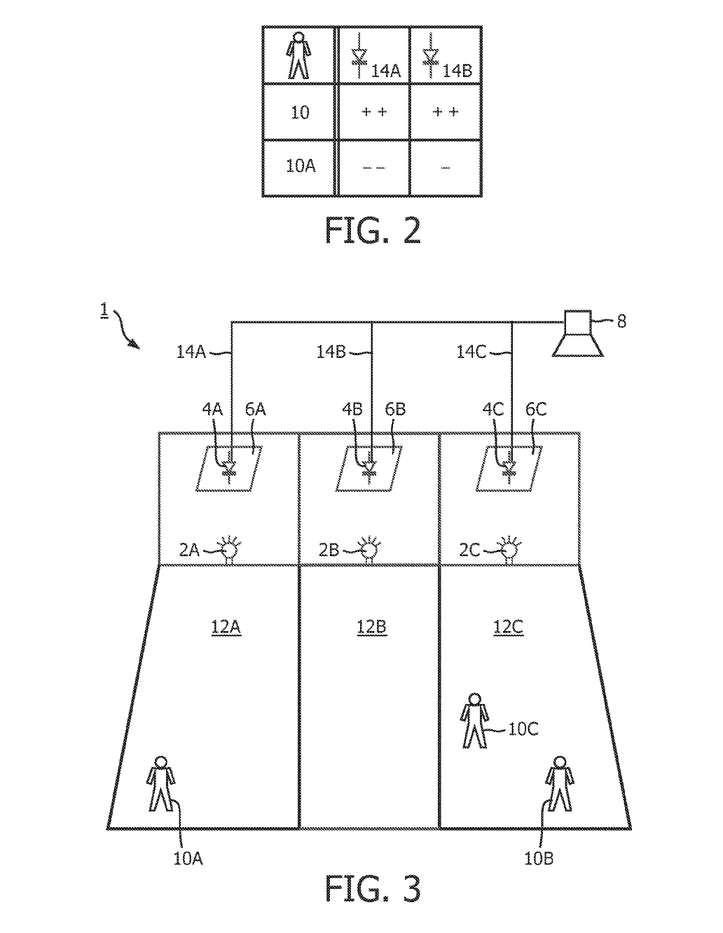

The light sources 2A;2B can also emit light not exclusively in one of the zones, and instead emit light of a relatively high intensity in a main zone and light of a relatively low intensity in the neighborhood zones of the main zone. Concretely, in the example shown in FIG. 1, the first light source 2A emits light of a relative high intensity in the first zone 12A and also light of a relative low intensity in the second zone 12B and similarly, the light source 2B emits light of a relative high intensity in the second zone 12B and also light of a relative low intensity in the first zone 12A. The light of the first light source 2A continually decreases from the main zone, in this example the first zone 12A, towards zones located more far away from the main zone. Such feature can be used to determine a relatively more precise position of the first human 10 within a zone. Particularly, in the example shown in FIG. 1, the heartbeat of the first human 10 will be detected by the first photo sensor 4A relatively strongly and relatively weakly by the second photo sensor 4B. However, for a second human 10A, shown in the same FIG. 1, the heartbeat of the second human 10A will be detected by the first photo sensor 4A as a relatively strong signal, approximately the same intensity as for the first human 10, and as a relatively weak signal detected by the second photo sensor 4B, but still with higher intensity compared to the first human 10. This is represented by the table shown in FIG. 2. As already mentioned, this feature can be used to determine a more precise position of the human within the zone. In this concrete example, the heartbeat measurements for the first human 10 and for the second human 10A will indicate that the second human 10A is closer to the second zone 12B than the first human 10, while it will be obvious that both humans are situated in the zone 12A.

The number of the zones within the area 12 can be different, actually any integer number higher than one. Increasing the number of the zones can increase the precision of the presence detection system. However, the number of the zones should be also selected in such a way that it suits the size of the area 12, namely a relatively small area can be very well covered by just two zones, while a relatively big area may require more than 20 zones.

FIG. 3 schematically shows a second exemplary embodiment of the presence detection system according to the invention, wherein the system is used to detect the presence of more than one human, in this concrete example the presence of three humans, a first human 10A, a second human 10B and a third human 10C. The processor device 8 will get three presence signals, a first presence signal 14A, a second presence signal 14B and a third presence signal 14C. These presence signals will indicate the presence of three humans. The first presence signal 14A will indicate the presence of the first human 10A having a first heartbeat rate. The third presence signal 14C will indicate the presence of the second human 10B and of the third human 10C having a second heartbeat rate and a third heartbeat rate respectively. This means that the system according to the invention is capable of detection of more than one human within different zones as well as within a single zone. This is possible because the presence signal comprises information about different heartbeats, detected from different humans, within one zone. In order to confirm that indeed two or more humans are present within the same zone it will be necessary to identify any difference in the measured heartbeats, for example different heart-rate, heart rate variability etc.

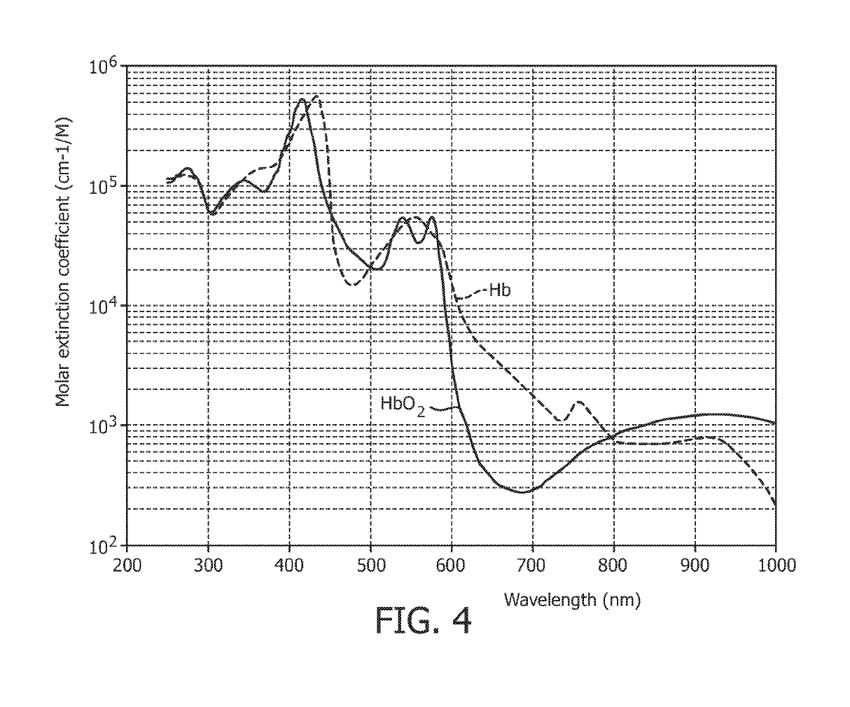

FIG. 4 shows the absorption spectrum of the oxhyhemoglobin (HbO₂) and deoxyhemoglobin (Hb) used for the measurement of the heart rate of the human. It is clear from FIG. 4 that measurement of the heart rate is possible using the light from a very broad range, at least from 200 to 1000 nanometers (nm).

FIG. 4 additionally shows that within the wavelength range from around 250 nm to around 1000 nm the light is strongly absorbed by the blood flow in the human's skin. As already said in the previous text that range corresponds to the ultraviolet spectrum, the infrared spectrum and the visible spectrum.

If the photodiode array is used, a one-time calibration of the system may be required in order to translate the spectrum color detected by the photodiode array into a spatial co-ordinate. However, as the illumination pattern is fixed and optionally matched 1:1 to the filters of the photo sensors, the calibration problem will be trivial.

The light sources can use light of non-visible wavelengths since it is not always desirable to illuminate the space with colored lighting. For this reason, a preferred embodiment of the invention proposes to construct the system whereby the spectrum bandwidth of the light source and the photosensor array filter fall outside the visible light wavelengths, i.e. 350-700 nanometers (nm).

The light is also strongly absorbed by the blood flow in the human's skin at both infrared (IR) wavelengths, above 700 nm, and at ultraviolet (UV) wavelengths, below 400 nm, as it is shown in FIG. 4. As the effect of UV light can be damaging for the skin, it is highly preferred that the spectrum frequencies which are used are IR frequencies where the light is still strongly absorbed by the blood flow in the skin. Such an IR based spectrum is highly suited as both IR light sources, for example LEDs, and photodiodes are readily available across the entire IR wavelength range.

The invention also proposes specific concepts whereby either the spectral bandwidth of the light source or the photodiode array filter is reduced. In general, the bandwidth of the light source should fall within the bandwidth of the photodiode array filter.

Furthermore, such a system may comprise a discrete or continuous light spectrum. It will be apparent to those experienced in the art that such a system may be created by using a broadband light spectrum or a series of light sources with reduced bandwidth spectrum, or with single frequency light sources, such as lasers and LEDs. Furthermore, it is also possible that each photodiode in the array has a discrete filter.

While the invention has been illustrated and described in detail in the drawings and foregoing description, such illustration and description are to be considered illustrative or exemplary and not restrictive; the invention is not limited to the disclosed embodiments.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measured cannot be used to advantage. Any reference signs in the claims should not be construed as limiting the scope.

### LIST OF REFERENCE NUMERALS

1 a presence detection system

2A;2B;2C a light source

4A;4B;4C a sensing means

6A;6B;6C a spectral filter

8 a processor device

10A;10B;10C a human

12A;12B;12C a zone

12 an area

14A;14B;14C a presence signal

1. A presence detection system for detecting a living being within an area comprising at least two zones, the system comprising:
at least two light sources each of them having a different predefined spectrum, a first spectrum and a second spectrum, and each of them radiating light in a different zone, and
at least two sensing means sensitive to light of the first predefined spectrum and the second predefined spectrum respectively, wherein each of the sensing means is arranged for detecting light reflected from the living being if present in the zone of the area and for generating a presence signal based on detected light,
a processor device for concluding the presence of the living being within the area based on the presence signals.

2. The presence detection system as claimed in claim 1, wherein the sensing means comprises a photo sensor sensitive to the light of the predefined spectrum.

3. The presence detection system as claimed in claim 1, wherein the sensing means comprises a photo sensor equipped with a spectral filter for filtering the light of predefined spectrum.

4. The presence detection system as claimed in claim 1, wherein the two light sources are comprised in a single light-radiation device, which the light-radiation device is arranged to radiate lights of at least two different predefined spectrums in two zones.

5. The presence detection system as claimed in claim 1, wherein the first spectrum and the second spectrum are substantially non-overlapping.

6. The presence detection system as claimed in claim 1, wherein the living being is a human and wherein the presence signal represents a human's vital signal.

7. The presence detection system as claimed in claim 6, wherein the presence signal represents the vital signals of at least two humans present in a same zone within the area and wherein the processor device distinguishes between the respective vital signals of the at least two humans present in the zone.

8. The presence detection system as claimed in claim 6, wherein the predefined spectrum of light emitted by the light sources is from the visible spectrum.

9. The presence detection system as claimed in claim 6, wherein the predefined spectrum of light emitted by the light sources is from the non visible spectrum.

10. The presence detection system as claimed in claim 6, wherein each of the photo sensors comprises a photodiode.

11. The presence detection system as claimed in claim 10, wherein the photodiodes are together arranged in a photodiode array.

12. A lighting system, comprising the presence detection system as claimed in claim 10 and a further light source for illuminating one or more of the zones or illuminating the area, wherein the presence detection systems is arranged for controlling the further light source based on the presence signals.
